(19) United States
(12) Patent Application Publication
(10) Pub. No.: US 2024/0076665 A1
(43) Pub. Date: Mar. 7, 2024

(54) REGULATORY ELEMENTS FOR SCHWANN CELL-SPECIFIC GENE EXPRESSION

(71) Applicant: Wisconsin Alumni Research Foundation, Madison, WI (US)

(72) Inventor: John Svaren, Madison, WI (US)

(73) Assignee: Wisconsin Alumni Research Foundation, Madison, WI (US)

(21) Appl. No.: 18/256,805

(22) PCT Filed: Dec. 22, 2021
(86) PCT No.: PCT/US2021/064820

§ 371 Date: Jun. 9, 2023

Related U.S. Application Data
(60) Provisional application No. 63/129,080, filed on Dec. 22, 2020.

(51) Int. Cl. C12N 15/113; C07K 14/47

(52) U.S. Cl. CPC C12N 15/113; C07K 14/4713; C12N 2310/14; C12N 2310/531; C12N 2320/32

(57) ABSTRACT

Regulatory elements that drive Schwann cell-specific gene expression, nucleic acid vectors, virus particles, and therapeutic compositions incorporating these constructs; and methods of using these various compositions to alter gene expression in a Schwann cell or to treat a subject having a condition associated with misexpression or insufficient function of a target gene in Schwann cells.

### CROSS-REFERENCE TO RELATED APPLICATIONS

Priority is hereby claimed to PCT application Serial No. PCT/US2021/064820, filed Dec. 22, 2021 (22.12.2021), which claims priority to U.S. application Ser. No. 63/129,080, filed Dec. 22, 2020 (22.12.2020), the contents of both of which are incorporated herein by reference.

### STATEMENT REGARDING FEDERALLY SPONSORED RESEARCH

This invention was made with government support under Grant Number R01 NS075269 awarded by the National Institutes of Health and the National Institute of Neurological Disorders and Stroke. The government has certain rights in this invention.

### BACKGROUND

Myelination of the peripheral nervous system involves activation of a genetic network in Schwann cells that coordinates formation of a multi-layer membrane sheath around axons. Disruptions of the integrity of this myelin sheath occurs in hereditary peripheral neuropathies (also known as hereditary motor sensory neuropathies, HMSN), which are among the most common genetic diseases. The mildest form of the disease, Charcot-Marie-Tooth (CMT) disease, causes progressive deterioration of both motor and sensory nerves, muscular atrophy, and chronic pain/fatigue in affected individuals. CMT is one of the most common forms of hereditary neurological disease, affecting 1 in 2500 individuals. As a result, more than 100,000 Americans are affected by inherited peripheral myelinopathies, and substantially more are affected by demyelination accompanying diabetic neuropathy and other disease states. Current treatment options are generally used to manage symptoms rather than effectively treating the disease itself.

A majority of inherited peripheral myelinopathies are caused by mutations of genes that affect the myelin coating of peripheral nerves that is produced by Schwann cells. Recently, gene therapy studies have used viral vectors to deliver gene replacement therapy in models of CMT1X and CMT4C, and studies are underway to deliver shRNA to reduce PMP22 expression since the most common form of CMT (CMT1A) is caused by a gene duplication of PMP22. For several types of demyelinating CMT, the responsible genes are specifically expressed in Schwann cells with cell-autonomous disease mechanisms, therefore restricting genetic therapies (either gene replacement or gene silencing) to Schwann cells is beneficial.

The development of a successful therapy depends upon finding an appropriate viral vector with the appropriate tropism to Schwann cells. Cell targeted expression is not only a matter of viral tropism but also a matter of promoter specificity. Expression of demyelinating neuropathy related genes has to be largely restricted to Schwann cells in order to avoid unpredictable effects in other cell types from ectopic gene expression. Thus, cell-specific promoters must be used. Since adeno-associated virus (AAV) vectors have limited transfer capacity, minimal myelin-specific promoters are needed in order to allow the packing of a number of neuropathy-associated genes. Cell-specific promoter elements are also needed to drive micro RNAs (shRNAs).

There are different promoters that can drive specific expression in glial cells such as the myelin protein zero (MPZ) (Sargiannidou et al., 2015; Scherer et al., 2005), the myelin basic protein (MBP) (Georgiou et al., 2017; von Jonquieres et al., 2013), the myelin associated glycoprotein (MAG) (von Jonquieres et al., 2016), the 2,3-cyclic nucleotide 3-phosphodiesterase (CNP) (Kagiava et al., 2014) and the glial fibrillary acidic protein (GFAP) (Xiang et al., 2018). The full-size myelin-specific rat MPZ promoter (Scherer et al., 2005) has been proven to drive long lasting expression in Schwann cells after intraneural (Sargiannidou et al., 2015) and intrathecal injection (Kagiava et al., 2016; Sargiannidou et al., 2015). However, many of these promoters are also expressed in other cell types like astrocytes and oligodendrocytes. For downregulation of PMP22, most existing technology uses ubiquitous promoters driving the shRNA. However, a stronger than expected downregulation of PMP22, or expression of shRNA in other cell types where it is not overexpressed, could be problematic. Accordingly, there remains a need in the art for constructs that drive Schwann cell-specific gene expression.

### SUMMARY

Disclosed herein is a nucleic acid construct comprising a Schwann cell-specific regulatory element, wherein the regulatory element is operably linked with a gene selected from the group consisting of myelin protein zero (MPZ), myelin associated glycoprotein (MAG), myelin basic protein (MBP), and apoptosis-associated tyrosine kinase (AATK).

In some versions of the construct, the regulatory element comprises at least a portion of a sequence selected from the groups consisting of SEQ ID NO: 1-6. In other versions, the regulatory element comprises the MAG enhancer of SEQ ID NO: 2 or SEQ ID NO: 3 or the minimal AATK promoter of SEQ ID NO: 6.

All of the versions of the nucleic acid construct may optionally comprise a peripheral myelin protein 22 (PMP22) P1 promoter. The Pmp22 P1 promoter may comprise SEQ ID NO: 7 or SEQ ID NO: 8.

It is generally preferred that the regulatory element is operably linked to a target gene (without limitation). The target gene may optionally be a short hairpin RNA (shRNA) that targets PMP22. The shRNA may comprise a sequence selected from SEQ ID NO: 9-17. While not required, the shRNA may be dimensioned, configured, and positioned within the construct to target selectively a single transcript isoform of PMP22.

It is preferred that the construct drives the expression of the target gene at an attenuated level in oligodendrocytes as compared to expression of the target gene in Schwann cells.

Also disclosed herein is a nucleic acid vector comprising any of the constructs disclosed herein.

Also disclosed herein is a virus particle comprising any of the constructs and vectors disclosed herein. The virus particle may be (but is not required to be) an adeno-associated virus particle.

Also disclosed herein is a therapeutic composition comprising the virus particle disclosed herein, optionally in combination with a pharmaceutically acceptable carrier.

Further disclosed herein is a method of altering gene expression in a Schwann cell, the method comprising delivering one or more of the constructs disclosed herein to a Schwann cell. The construct may be delivered by a virus particle.

Also disclosed herein is a method of treating a subject having a condition associated with misexpression or attenuated function of a target gene in Schwann cells. The method comprises administering a therapeutically effective amount of a construct disclosed herein, and/or a virus particle disclosed herein, and/or the therapeutic composition disclosed herein to a subject. The subject may be a mammal. The subject may be a human being. The method may be administered to treat, to ameliorate, or to inhibit the onset or progress of a peripheral neuropathy. Such peripheral neuropathies include, but are not limited to, Charcot-Marie-Tooth disease, Guillain-Barré syndrome, schwannomatosis, neurofibromatosis, chronic inflammatory demyelinating polyneuropathy, and leprosy. For example, the condition may be Charcot-Marie-Tooth disease type 1A (CMT1A), and the target gene is a shRNA that targets PMP22. Or the condition is the X-linked form of Charcot-Marie-Tooth disease (CMT1X) and the target gene is gap junction beta 1 (GJB1). Or the condition is Charcot-Marie-Tooth neuropathy type 4C (CMT4C) and the target gene is SH3 domain and tetratricopeptide repeats 2 (SH3TC2).

The virus particle or therapeutic composition disclosed herein may be administered intravenously, intraneurally, or intrathecally. It is generally preferred (although not required) that between 10⁹ and 10¹² copies of the virus particle are administered to the subject. As noted previously, the subject may be a mammal, including humans.

Specifically disclosed and claimed herein are the following:
1. A nucleic acid construct comprising a Schwann cell-specific regulatory element, wherein the regulatory element is operably linked with a gene selected from the group consisting of myelin protein zero (MPZ), myelin-associated glycoprotein (MAG), myelin basic protein (MBP), and apoptosis-associated tyrosine kinase (AATK).
2. The nucleic acid construct of claim 1, wherein the regulatory element comprises at least a portion of a sequence selected from the groups consisting of SEQ ID NO:1-6.
3. The nucleic acid construct of claim 2, wherein the regulatory element is the MAG enhancer of SEQ ID NO:2 or SEQ ID NO:3 or is the minimal AATK promoter of SEQ ID NO:6.
4. The nucleic acid construct of claim 1, further comprising a peripheral myelin protein 22 (PMP22) P1 promoter.
5. The nucleic acid construct of claim 4, wherein the Pmp22 P1 promoter comprises SEQ ID NO:7 or SEQ ID NO:8.
6. The nucleic acid construct of claim 4, wherein the regulatory element comprises at least a portion of a sequence selected from the groups consisting of SEQ ID NO:1-6.
7. The nucleic acid construct of claim 4, wherein the regulatory element is the MAG enhancer of SEQ ID NO:2 or SEQ ID NO:3 or is the minimal AATK promoter of SEQ ID NO:6.
8. The nucleic acid construct of claim 1, wherein the regulatory element is operably linked to a target gene.
9. The nucleic acid construct of claim 8, further comprising a peripheral myelin protein 22 (PMP22) P1 promoter.
10. The nucleic acid construct of claim 9, wherein the Pmp22 P1 promoter comprises SEQ ID NO:7 or SEQ ID NO:8.
11. The nucleic acid construct of claim 8, wherein the regulatory element comprises at least a portion of a sequence selected from the groups consisting of SEQ ID NO:1-6.
12. The nucleic acid construct of claim 8, wherein the regulatory element is the MAG enhancer of SEQ ID NO:2 or SEQ ID NO:3 or is the minimal AATK promoter of SEQ ID NO:6.
13. The nucleic acid construct of claim 8, wherein the target gene is a short hairpin RNA (shRNA) that targets PMP22.
14. The nucleic acid construct of claim 13, wherein the shRNA comprises a sequence selected from SEQ ID NO:9-17.
15. The nucleic acid construct of claim 8, wherein the shRNA is dimensioned, configured, and positioned within the construct to target selectively a single transcript isoform of PMP22.
16. The nucleic acid construct of claim 8, wherein the construct drives expression of the target gene at an attenuated level in oligodendrocytes as compared to expression of the target gene in Schwann cells.
17. A nucleic acid vector comprising the nucleic acid construct of claim 1.
18. A nucleic acid vector comprising the nucleic acid construct of claim 8.
19. A virus particle comprising the nucleic acid construct of claim 1.
20. A virus particle comprising the nucleic acid construct of claim 8.
21. A method of altering gene expression in a Schwann cell, the method comprising delivering the nucleic acid construct as recited in claim 1 or claim 8 to the Schwann cell.
22. The method of claim 21, wherein the nucleic acid construct is delivered by a virus particle.
23. A method of treating a subject having a condition associated with misexpression or attenuated function of a target gene in Schwann cells, the method comprising administering a therapeutically effective amount of a nucleic acid construct as recited in claim 1 or claim 8 to the subject.
24. The method of claim 23, wherein the condition is a peripheral neuropathy.
25. The method of claim 24, wherein the condition is selected from the group consisting of Charcot-Marie-Tooth disease, Guillain-Barré syndrome, schwannomatosis, chronic inflammatory demyelinating polyneuropathy, and leprosy.
26. The method of claim 24, comprising administering a therapeutically effective amount of a nucleic acid construct as recited in claim 8 to the subject, wherein the condition is Charcot-Marie-Tooth disease type 1A (CMT1A), and wherein the target gene is a shRNA that targets PMP22.
27. The method of claim 24, comprising administering a therapeutically effective amount of a nucleic acid construct as recited in claim 8 to the subject, wherein the condition is the X-linked form of Charcot-Marie-Tooth disease (CMT1X), and wherein the target gene is gap junction beta 1 (GJB1).
28. The method of claim 24, comprising administering a therapeutically effective amount of a nucleic acid construct as recited in claim 8 to the subject, wherein the condition is Charcot-Marie-Tooth neuropathy type 4C (CMT4C), and wherein the target gene is SH3 domain and tetratricopeptide repeats 2 (SH3TC2).

### BRIEF DESCRIPTION OF THE DRAWINGS

FIG. 1 is a gene map depicting a 1.4 Mb duplication containing the peripheral myelin protein 22 (PMP22) gene, which is the most common cause of Charcot-Marie-Tooth Disease type 1A (CMT1A).

FIG. 2 is a graph depicting the results of a luciferase assay in which several constructs were tested for preferential activation in Schwann cells (S16) as compared to oligodendrocytes (Oli neu). The constructs comprised the indicated enhancer cloned upstream of a minimal promoter (E1B) or a Schwann cell-specific promoter (Pmp22 P1 or Mpz).

FIG. 3 is a graph depicting the results of a luciferase assay in which several constructs were tested for preferential activation in Schwann cells (RT4) as compared to oligodendrocytes (Oli neu). For this assay, several putative Schwann cell-specific regulatory elements (i.e., Mag, Mp11, Mbp, Pmp22) were cloned upstream of the Schwann cell-specific, full-length Pmp22 P1 promoter.

FIG. 4 is a graph depicting the results of a luciferase assay in which several constructs were tested for preferential activation in Schwann cells (RT4) as compared to oligodendrocytes (Oli neu). For this assay, Mag and Mbp regulatory elements were cloned upstream of both the full-length Pmp22 P1 promoter (P1) and a shorter version of this promoter (delta2).

FIGS. 5A and 5B are graphs depicting the results of a luciferase assay in which constructs comprising several different versions of the Aatk promoter were tested for activation in Schwann cells (RT4). For comparison, the delta2 P1 promoter of PMP22 was also included. FIG. 5A shows the results in luciferase activity. FIG. 5B shows the results in terms of fold-change in luciferase activity.

FIG. 6 is a schematic diagram of adeno-associated virus (AAV) vector constructs comprising the Schwann cell-specific regulatory elements disclosed herein.

FIG. 7 is a graph depicting the results of a luciferase assay in which constructs comprising shRNA with 5′ UTR no. 1 (sh51) were tested for activation in Schwann cells (RT4) with shRNA1 and scrambled shRNA (scr) as controls.

FIG. 8 is a graph depicting the results of qPCR assay measuring total PMP22 and its two major transcripts (P1 and P2) in human Schwann cells with Mpz pro/int vectors expressing sh51, shRNA1 or scr.

FIG. 9 is a graph depicting the results of a luciferase assay in which constructs comprising 3 different shRNAs (sh51, sh52, and sh53) were tested for activation in Schwann cells (RT4).

FIG. 10 is a graph depicting the results of a luciferase assay in which several constructs comprising sh53 were tested for activation in Schwann cells (RT4).

### DETAILED DESCRIPTION

Disclosed herein are a series of Schwann cell-specific regulatory elements. These regulatory elements can be used in gene therapies that correct the mis-regulation of a gene that is expressed in Schwann cells. The inventors have designed constructs comprising these regulatory elements that drive a range of gene expression levels, thereby allowing such therapies to be tailored to produce an appropriate level of the therapeutic gene product. These regulatory elements preferentially allow for expression in Schwann cells as opposed to other cell types, e.g. motor neurons, and thus allows for specific targeting to Schwann cells.

While the use of gene therapies for the treatment of motor neuron diseases is well established (e.g., for spinal muscular atrophy), intrathecal injection of viral gene therapy vectors results in the transduction of several cell types within the central nervous system (CNS). Thus, the constructs of the present invention have been designed to drive expression at high levels in Schwann cells, but at minimal levels in other cell types, thereby avoiding side effects caused by off-target expression of the therapeutic gene product.

### Abbreviations and Definitions

The articles “a” and “an” are used herein to refer to one or more than one (i.e., to at least one of the grammatical object of the article. By way of example, “an element” means one element or more than one element.

The term “about” is used herein to mean a value ±10% of the given numerical value.

The term “alignment” refers to a method of comparing, two or more polynucleotides or polypeptide sequences for the purpose of determining their relationship to each other. Alignments are typically performed by computer programs that apply various algorithms, however it is also possible to perform an alignment by hand. Alignment programs typically iterate through potential alignments of sequences and score the alignments using substitution tables, employing a variety of strategies to reach a potential optimal alignment score. Commonly-used alignment algorithms include, but are not limited to, CLUSTALW, (see, Thompson J. D., Higgins D. G., Gibson T. J., CLUSTAL W: improving the sensitivity of progressive multiple sequence alignment through sequence weighting, position-specific gap penalties and weight matrix choice, Nucleic Acids Research 22: 4673-4680, 1994); CLUSTALV, (see, Larkin M. A., et al., CLUSTALW2, ClustalW and ClustalX version 2, Bioinformatics 23(21): 2947-2948, 2007); Jotun-Hein, Muscle et al., MUSCLE: a multiple sequence alignment method with reduced time and space complexity, BMC Bioinformatics 5: 113, 2004); Mafft, Kalign, ProbCons, and T-Coffee (see Notredame et al., T-Coffee: A novel method for multiple sequence alignments, Journal of Molecular Biology 302: 205-217, 2000). Exemplary programs that implement one or more of the above algorithms include, but are not limited to MegAlign®-brand software from DNAStar (DNAStar, Inc. Madison, Wisconsin), MUSCLE, T-Coffee, CLUSTALX, CLUSTALV, JalView, Phylip, and Discovery Studio from Accelrys (Accelrys, Inc. San Diego, California). In a non-limiting example, the MegAlign®-brand software was used to implement the CLUSTALW alignment algorithm with the following parameters: Gap Penalty 10, Gap Length Penalty 0.20, Delay Divergent Seqs (30%) DNA Transition Weight 0.50, Protein Weight matrix Gonnet Series, DNA Weight Matrix IUB.

The term “attenuate” means to weaken, reduce or diminish.

The term “consensus sequence” or “canonical sequence” refers to an archetypical amino acid sequence against which all variants of a particular protein or sequence of interest are compared. Either term also refers to a sequence that sets forth the nucleotides that are most often present in a polynucleotide sequence of interest. For each position of a protein, the consensus sequence gives the amino acid that is most abundant in that position in the sequence alignment.

The term “conservative substitutions” or “conserved substitutions” refers to, for example, a substitution wherein one or more of the following amino acid substitutions are made: replacement of an aliphatic amino acid, such as alanine, valine, leucine, and isoleucine, with another aliphatic amino acid; replacement of a serine with a threonine; replacement of a threonine with a serine; replacement of an acidic residue, such as aspartic acid and glutamic acid, with another acidic residue; replacement of a residue bearing an amide group, such as asparagine and glutamine, with another residue bearing an amide group; exchange of a basic residue, such as histidine, lysine and arginine, with another basic residue; and replacement of an aromatic residue, such as tryptophan, phenylalanine and tyrosine, with another aromatic residue; or replacement of small amino acids, such as glycine, alanine, serine, threonine and methionine, with another small amino acid. Amino acid substitutions which do not generally alter the specific activity are known in the art and are described, for example, by H. Neurath and R. L. Hill, (Eds.) in “The Proteins, Third Edition” Academic Press, New York, © 1979. Useful conservative modifications include Alanine to Cysteine. Glycine, or Serine; Arginine to Isoleucine, Lysine, Methionine. or Ornithin; Asparagine to Aspartic acid, Glutamine, Glutamic acid, or Histidine; Aspartic acid to Asparagine, Glutamine, or Glutamic acid; Cysteine to Methionine, Serine, or Threonine; Glutamine to Asparagine, Aspartic acid, or Glutamic acid; Glutamic acid to Asparagine, Aspartic acid, or Glatmine; Glycine to Aspartic acid, Alanine, or Proline; Histidine to Asparagine, or Glutamine; Isoleucine to Leucine, Methionine, or Valine; Leucine to Isoleucine, Methionine, or Valine; Lysine to Arginine, Glutamine, Glutamic acid, Isoleucine, Methionine, or Ornithin; Methionine to Cysteine, Isoleucine, Leucine, or Valine; Phenylalanine to Histidine, L-Dopa, Leucine, Methionine, Threonine, Tryptophan, Tyrosine, 3-phenylproline, 4-phenylproline, or 5-phenylproline; Proline to L-1-thioazolidine-4-carboxylic acid or D- or L-1-oxazolidine-4-carboxylic acid; Serine to Cysteine, Methionine, or Threonine; Threonine to Methionine, Serine, or Valine; Tryptophan to Tyrosine; Tyrosine to L-Dopa, Histidine, or Phenylalanine; and Valine to Isoleucine, Leucine, or Methionine.

A polynucleotide is said to “encode” an RNA or a polypeptide if, in its native state or when manipulated by methods known to those of skill in the art, it can be transcribed and/or translated to produce the corresponding RNA, the corresponding polypeptide, or a fragment thereof. The antisense strand of such a polynucleotide is also said to encode the RNA or polypeptide sequences. As is known in the art, a DNA can be transcribed by an RNA polymerase to produce an RNA, and an RNA can be reverse transcribed by reverse transcriptase to produce a DNA. Thus, a DNA can encode an RNA, and vice versa.

“Gene” refers to a polynucleotide (e.g., a DNA segment), which encodes a polypeptide, and includes regions preceding and following the coding regions as well as intervening sequences (introns) between individual coding segments (exons).

The term “homologous genes” refers to a pair of genes from different but related species, which correspond to each other, and which are identical or similar to each other. The term encompasses genes that are separated by the speciation process during the development of new species (e.g., orthologous genes), as well as genes that have been separated by genetic duplication (e.g., paralogous genes).

“Homology” refers to sequence similarity or sequence identity. Homology is determined using standard techniques known in the art (see, e.g., Smith and Waterman, Adv. Appl. Math., 2:482, 1981; Needleman and Wunsch, J. Mol. Biol., 48:443, 1970; Pearson and Lipman, Proc. Natl. Acad. Sci. USA 85:2444, 1988; programs such as GAP, BESTFIT, FASTA, and TFASTA in the Wisconsin Genetics Software Package (Genetics Computer Group, Madison. Wisconsin); and Devereux et al., Nucl. Acid Res., 12:387-395, 1984). A non-limiting example includes the use of the BLAST program (Altschul et al., Gapped BLAST and PSI-BLAST: a new generation of protein database search programs. Nucleic Acids Res. 25:3389-3402, 1997) to identify sequences that can be said to be “homologous.” A recent version such as version 2.2.16, 2.2.17, 2.2.18, 2.2.19, or the latest version, including sub-programs such as blastp for protein-protein comparisons, blastn for nucleotide-nucleotide comparisons, tblastn for protein-nucleotide comparisons, or blastx for nucleotide-protein comparisons, and with parameters as follows: Maximum number of sequences returned 10,000 or 100,000; E-value (expectation value) of 1e-2 or 1e-5, word size 3, scoring matrix BLOSUM62, gap cost existence 11, gap cost extension 1, may be suitable. An E-value of 1e-5, for example, indicates that the chance of a homologous match occurring at random is about 1 in 10,000, thereby marking a high confidence of true homology.

The term “host strain” or “host cell” refers to a suitable host for an expression vector comprising a nucleic acid construct as described herein.

The term “operably linked,” in the context of a polynucleotide sequence, refers to the placement of one polynucleotide sequence into a functional relationship with another polynucleotide sequence. For example, a DNA encoding a secretory leader (e.g., a signal peptide) is operably linked to a DNA encoding a polypeptide if it is expressed as a preprotein that participates in the secretion of the polypeptide. A promoter or an enhancer is operably linked to a coding sequence if it affects the transcription of the sequence. A ribosome binding site is operably linked to a coding sequence if it is positioned to facilitate translation. “Operably linked” DNA sequences need not be contiguous, although they may be.

The terms “percent sequence identity,” “percent amino acid sequence identity,” “percent gene sequence identity,” and/or “percent polynucleotide sequence identity,” with respect to two polypeptides, polynucleotides and/or gene sequences (as appropriate), refer to the percentage of residues that are identical in the two sequences when the sequences are optimally aligned. Thus, 80% amino acid sequence identity means that 80% of the amino acids in two optimally aligned polypeptide sequences are identical.

The term “selectable marker” or “selective marker” refers to a polynucleotide (e.g., a gene) capable of expression in a host cell, which allows for ease of selection of those hosts containing the vector. Examples of selectable markers include but are not limited to antimicrobial markers. Thus, the term “selectable marker” refers to a gene that provides an indication when a host cell has taken up an incoming sequence of interest or when some other reaction has taken place. Typically, selectable markers are genes that confer antimicrobial resistance or a metabolic advantage on the host cells to allow the cells containing the exogenous sequences to be distinguished from the cells that have not received the exogenous sequences. A “residing selectable marker” is one that is located on the chromosome of the microorganism to be transformed. A residing selectable marker encodes a gene that is different from the selectable marker on the transforming construct. Selective markers are known to those of skill in the art. As indicated above, suitably the marker is an antimicrobial resistant marker, including, for example, ampR; phleoR; specR; kanR; eryR; tetR; cmpR; and neoR. See. e.g., Guerot-Fleury, Gene, 167:335-337, 1995; Palmeros et al., Gene, 247:255-264, 2000; and Trieu-Cuot et al., Gene, 23:331-341, 1983. Other markers useful include, but are not limited to, auxotrophic markers, such as tryptophan; and detection markers, such as 6-galactosidase.

### Constructs:

Disclosed herein are constructs comprising a Schwann cell-specific regulatory element, wherein the regulatory element is operably linked with a gene selected from the group consisting of myelin protein zero (MPZ), myelin associated glycoprotein (MAG), myelin basic protein (MBP), and apoptosis-associated tyrosine kinase (AATK).

As used herein, the term “construct” refers to an artificially constructed segment of nucleic acid. The constructs of the present invention comprise at least one Schwann cell-specific regulatory element, and more specifically to at least one Schwann cell-specific regulatory element operably linked to a gene of interest to be expressed in Schwann cells. As used herein, the term “regulatory element” refers to a segment of DNA that is dimensioned and configured to regulate the transcription of specific genes. Specifically, the regulatory elements used in the constructs of the disclosed herein comprise enhancer and/or promoter elements that drive the expression of an operably linked target gene. The term “promoter” typically refers to a regulatory region that is capable of binding RNA polymerase in a cell and initiating transcription of a downstream coding sequence, whereas the term “enhancer” typically refers to a regulatory region that is bound by other proteins that promote transcription (i.e., transcription factors). However, in practice, the traditional distinction between these two types of regulatory elements is often blurred.

In some embodiments, the regulatory element is operably linked to a target gene within the construct. Preferably, the target gene encodes a therapeutic gene product that can be used to treat a condition associated with misexpression or insufficient function of a target gene in Schwann cells. For example, the target gene may encode a functional protein that can be used to replace a protein that is dysfunctional or is expressed at an insufficient level due to a genetic mutation (i.e., a gene replacement therapy). Alternatively, the target gene may encode a negative regulator, such as a short hairpin RNA (shRNA), that reduces that expression of a misexpressed gene (i.e., a gene knockdown therapy).

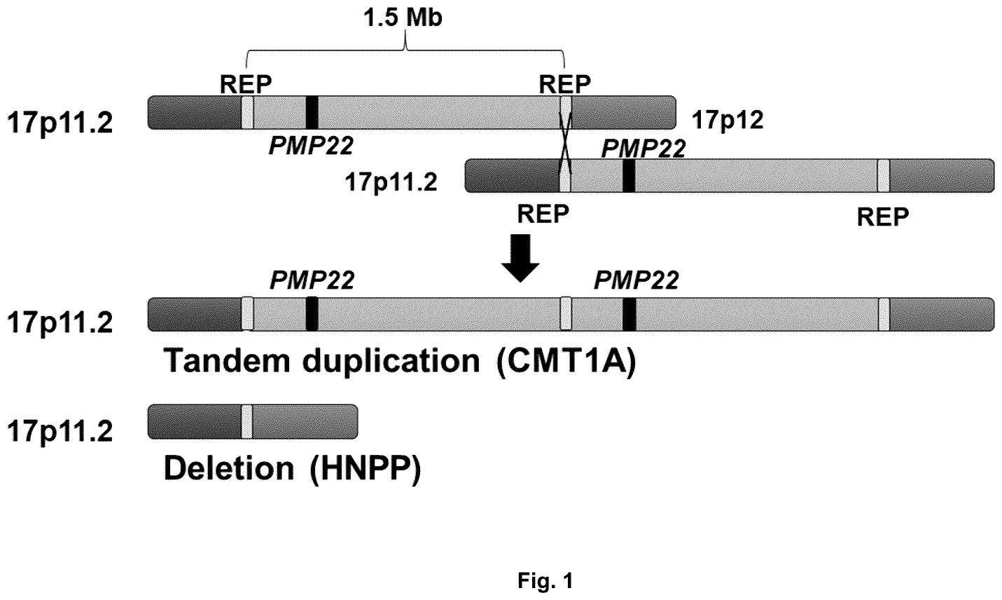

For example, in some embodiments, the construct is designed to treat Charcot-Marie-Tooth disease type 1A (CMT1A) by driving the expression of a shRNA that targets PMP22, e.g., the shRNA of SEQ ID NOS: 9-17. PMP22 is overexpressed in CMT1A due to a 1.4 Mb gene duplication (see FIG. 1). Importantly, because underexpression of PMP22 causes another peripheral nerve disorder (i.e., hereditary neuropathy with liability to pressure palsy, which is often caused by a deletion of the PMP22 gene), the inventors have designed shRNAs that specifically target only one of the two PMP22 transcript isoforms (i.e., 1a or 1b) that are abundant in the sural nerve. Because the PMP22 transcript isoforms are expressed in a 1:1 ratio, targeting a single isoform maximally reduces PMP22 expression by 50%, avoiding the risk of reducing the expression of PMP22 by too much. Thus, in some embodiments, the shRNA selectively targets a single transcript isoform of PMP22. In specific embodiments, the shRNA comprises a sequence selected from SEQ. ID. NOS: 9-17 or SEQ ID NOS: 12-14.

The primary advantage of the construct disclosed herein is that the regulatory elements are “Schwann cell-specific”, meaning they drive gene expression at high levels in Schwann cells, but at minimal levels in other cell types. This design reduces the risk for off-target effects that could result from expression of a target gene in other cell types. In the Examples, the inventors tested the cell-type specificity of their constructs by transfecting them into both Schwann cells and oligodendrocytes and compared their relative activity in these cell types using a luciferase assay. Like Schwann cells, oligodendrocytes are myelinating glia cells, and these cell types share some transcriptional mechanisms. For example, the same regulatory elements are used to drive expression of particular myelin genes that are expressed in both Schwann cells and oligodendrocytes. Thus, of all the cell types in the central nervous system, oligodendrocytes are the most likely candidate for off-target expression of the constructs disclosed herein. Thus, in preferred embodiments, the constructs drive the expression of the target gene at a minimal level in oligodendrocytes.

To generate the constructs, the inventors have selected regulatory elements that are associated with the genes myelin protein zero (MPZ), myelin associated glycoprotein (MAG), myelin basic protein (MBP), and apoptosis-associated tyrosine kinase (AATK). The inventors initially identified these putative Schwann cell-specific regulatory elements using a ChIP-seq data set comprising active enhancer marks (e.g., histone H3K27 acetylation) and transcription factor binding sites (e.g., SOX10 and EGR2) in rat sciatic nerve cells. (Regarding ChIP-sequencing itself, see, for example, Jung et al. (May 2014) “Impact of sequencing depth in ChIP-seq experiments,” Nucleic Acids Research 42 (9): e74.) To create the constructs disclosed herein, the inventors modified and/or combined these regulatory elements to achieve a desired level of Schwann cell-specific gene expression. For example, in one embodiment, the inventors have combined a regulatory element from the MAG gene (for example, SEQ ID NO: 2 or SEQ ID NO: 3) and a promoter from the PMP22 gene (for example, SEQ ID NO: 7 or SEQ ID NO: 8). In another embodiment, the inventors have combined an intronic enhancer and a promoter from the MPZ gene (a non-limiting example of which is shown in SEQ ID NO: 1). In a third embodiment, the inventors have combined a small, highly conserved region of the AATK gene comprising an intronic binding sequence for the transcription factor Sox10 with the AATK promoter (a non-limiting example of which is shown in SEQ ID NO: 6). Thus, in some embodiments, the regulatory element comprises at least a portion of a sequence selected from the groups consisting of SEQ ID NO: 1-6.

One of the disclosed regulatory elements, i.e., the minimal AATK promoter, has been shown by the present inventors to drive Schwann cell-specific expression of a miRNA, which suggests that it should also drive the expression of shRNAs. Thus, in some embodiments, the regulatory element is the minimal AATK promoter of SEQ ID NO: 6.

In the Examples, the inventors cloned the disclosed regulatory elements upstream of a promoter from the gene peripheral myelin protein 22 (PMP22) to test their ability to drive Schwann cell-specific gene expression in a luciferase assay. Thus, in some embodiments, the constructs further comprise PMP22 P1 promoter. In specific embodiments, the PMP22 P1 promoter comprises the full-length PMP22 P1 promoter (SEQ ID NO: 7), and in others it comprises a minimal PMP22 P1 promoter (SEQ ID NO: 8).

### Vectors, Viruses, and Therapeutic Compositions:

As used herein, the term “vector” refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of, and are dimensioned and configured to direct the expression of nucleic acids to which they are operatively linked. Such vectors are referred to herein as “expression vectors”. Vectors suitable for use herein comprise the Schwann-cell specific regulatory elements described herein, a target gene of interest and a heterogeneous sequence necessary for proper propagation of the vector and expression of any encoded target gene.

Also disclosed herein are virus particles comprising the present constructs or nucleic acid vectors. Suitable viruses are known in the art and include, but are not limited to, adenovirus, adeno-associated virus, lentivirus, fowlpox virus, alpha virus, baculovirus, and herpes virus.

Adeno-associated viruses have the ability to pass through the blood brain barrier to reach target cells, making them suitable for the treatment of neuropathies. Thus, in a preferred embodiment, the virus particle is an adeno-associated virus particle. Notably, in such embodiments, the use of minimal promoters may be advantageous, as adeno-associated virus vectors have a limited transfer capacity.

Also disclosed herein are therapeutic compositions comprising the virus particles described herein and a pharmaceutically acceptable carrier. “Pharmaceutically acceptable” carriers are known in the art and include, but are not limited to, suitable diluents, preservatives, solubilizers, emulsifiers, liposomes, nanoparticles, and adjuvants. Pharmaceutically acceptable carriers may be aqueous or nonaqueous solutions, suspensions, and emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions or suspensions, including saline and buffered media.

The therapeutic compositions of the present invention may further comprise liquids or lyophilized or otherwise dried formulations and may include diluents of various buffer content (e.g., Tris-HCl, acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polyglycolic acid, hydrogels, etc, or onto liposomes, microemulsions, micelles, milamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils).

### Methods:

Disclosed herein are methods of altering gene expression in a Schwann cell. The methods comprise delivering the nucleic acid constructs disclosed herein to a Schwann cell. These methods can be performed in vivo or ex vivo (e.g., as a therapeutic treatment) or in vitro (e.g., for research applications). These methods may be used to increase or decrease the expression of a particular target gene. For in vivo and ex vivo applications, the construct may be delivered to the Schwann cell by a virus particle, e.g., via administration of a virus particle comprising the constructs described herein. In some embodiments, the virus particle may be injected directly into a peripheral nerve to reduce infection of other cell types within the central nervous system. For in vitro applications, the construct may be delivered via transfection. Transfection methods are well known in the art and include, for example, electroporation, calcium phosphate exposure, and liposome-based transfections.

In another aspect, the method comprises treating a subject having a condition associated with misexpression or insufficient function of a target gene in Schwann cells. The methods compriss administering a therapeutically effective amount of a construct, virus particle, or therapeutic composition described herein to the subject.

In some embodiments, the constructs are delivered to Schwann cells using the virus particles described herein. In other embodiments, the constructs are delivered to Schwann cells using another drug delivery system that can function as an exogenous DNA carrier, such as nanoparticles, extracellular vesicles, or exosomes.

Schwann cells are involved in many important aspects of peripheral nerve biology—the conduction of nervous impulses along axons, nerve development and regeneration, trophic support for neurons, production of the nerve extracellular matrix, modulation of neuromuscular synaptic activity, and presentation of antigens to T-lymphocytes. As a result, Schwann cell dysfunction can cause a variety of peripheral neuropathies, i.e., conditions that result when nerves that carry messages to and from the brain and spinal cord to the rest of the body are damaged or diseased. Thus, in some embodiments, the condition that is treated by the present methods is a peripheral neuropathy. Suitably, the peripheral neuropathy is one that involves Schwann cells, such as Charcot-Marie-Tooth (CMT) disease, Guillain-Barré syndrome, schwannomatosis, chronic inflammatory demyelinating polyneuropathy, or leprosy. Alternatively, the methods may be used to treat neurofibromatosis, an inherited, Schwann cell-derived cancer. Here, the constructs can be designed to express a shRNA that targets an oncogenic gene (e.g., Sox9) or restores the function of a tumor suppressor gene (e.g., NF1, NF2, EED, SUZ12). The disclosed methods may also be used to treat diabetic neuropathy, e.g., by targeting the aldose reductase gene to reduce sorbitol levels.

For several types of demyelinating Charcot-Marie-Tooth (CMT) disease, the responsible genes are specifically expressed in Schwann cells. Thus, restricting such genetic therapies to Schwann cells would be beneficial. For example, as discussed above, Charcot-Marie-Tooth disease type 1A (CMT1A) is caused by overexpression of the gene PMP22 in Schwann cells. Thus, in one particular embodiment, the condition is CMT1A and the target gene is a shRNA that targets PMP22. The X-linked form of Charcot-Marie-Tooth disease (CMT1X) is caused by hundreds of different mutations in the gene gap junction beta 1 (GJB1), which result in impaired gap junction formation between myelinating cells. Thus, in another embodiment, the condition is CMT1X and the target gene is GJB1. Charcot-Marie-Tooth neuropathy type 4C (CMT4C) is caused by loss-of-function mutations in the gene SH3 domain and tetratricopeptide repeats 2 (SH3TC2). Thus, in a third embodiment, the condition is CMT4C and the target gene is SH3TC2.

As used herein, “treating” or “treatment” describes the management and care of a subject for the purpose of combating a disease, condition, or disorder. Treating includes the administration of a construct, vector, virus, or composition as disclosed herein to prevent, ameliorate, attenuate, or otherwise reduce the onset of the symptoms or complications, to alleviate the symptoms or complications, or to eliminate, attenuate, or slow the progression of the disease, condition, or disorder. Specifically, the compositions disclosed herein can be used to treat subjects suffering from a condition associated with misexpression or insufficient function of a target gene in Schwann cells. Treatment may result in reduction of one or more symptoms associated with the neuropathy condition described herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, intradermal administration, intrathecal administration, and subcutaneous administration. Administration can be continuous or intermittent. Single or multiple administrations can be carried out. In preferred embodiments of the present methods, the virus particle or therapeutic composition is administered intravenously, intraneurally, or intrathecally.

The term “therapeutically effective amount” refers to an amount that is sufficient to effect beneficial or desirable biological or clinical results. That result can be reducing, alleviating, inhibiting, or preventing one or more symptoms of a disease or condition, or can be any other desired alteration of a biological system.

In one embodiment, between 10⁹ and 10¹² copies of the virus particle are administered to the subject. However, the appropriate dosage will vary with the formulation used for therapy, the purpose of the therapy, the method of administration, and the subject being treated. Methods for determining an effective dosage are well known to those of skill in the art.

As used herein, the term “subject” refers to mammals and non-mammals. A “mammal” may be any member of the class Mammalia including, but not limited to, humans, non-human primates (e.g., chimpanzees, other apes, and monkey species), farm animals (e.g., cattle, horses, sheep, goats, and swine), domestic animals (e.g., rabbits, dogs, and cats), or laboratory animals including rodents (e.g., rats, mice, and guinea pigs). Examples of non-mammals include, but are not limited to, birds, and the like. The term “subject” does not denote a particular age or sex. In one embodiment, the subject is a human. In a preferred embodiment, the human has a Schwann cell-related peripheral neuropathy.

The use herein of the terms “including,” “comprising,” or “having,” and variations thereof, is meant to encompass the elements listed thereafter and equivalents thereof as well as additional elements. Embodiments recited as “including,” “comprising” or “having” certain elements are also contemplated as “consisting essentially of” and “consisting of” those certain elements.

The present invention has been described in terms of one or more preferred embodiments, and it should be appreciated that many equivalents, alternatives, variations, and modifications, aside from those expressly stated, are possible and within the scope of the invention.

### EXAMPLES

The following Example describes the inventors' efforts to design and test regulatory elements that drive Schwann cell (SC)-specific gene expression.

### Screening of Regulatory Elements for Schwann Cell-Specific Expression In Vitro:

The ability of the new constructs to drive expression in a Schwann cell-specific manner is tested by transfecting the constructs into Schwann cell lines S16 (CRL-2941) and RT4 (CRL-2768) (American Type Culture Collection, Manassas, Virginia) and related myelinating glia known as oligodendrocytes (i.e., Oli-neu; RRID:CVCL_IZ82; Jung M, Krämer E, Grzenkowski M, Tang K, Blakemore W, Aguzzi A, Khazaie K, Chlichlia K, von Blankenfeld G, Kettenmann H (1995) Eur J Neurosci. 7(6):1245-65) and comparing their relative activity in these cell types. For preliminary testing, the selected regulatory elements were cloned into pGL3 or pGL4 luciferase reporter vectors (Promega, Fitchburg, Wisconsin, USA). The E1B TATA element was initially used as a minimal promoter, but a full-length and short version of the Pmp22 P1 promoter have also been tested (SEQ ID NO: 7 and SEQ ID NO: 8, respectively). Transfections were performed as previously described (Jones et al., 2012; Srinivasan et al., 2012), using co-transfection with the herpes simplex virus thymidine kinase (HSV-TK) promoter-driven Renilla luciferase (Promega) to normalize reporter activity between experiments. Promoter activity was measured 48 hours post-transfection.

### Design of Schwann Cell-Specific Constructs:

### Mag/Mbp and Mpz Regulatory Elements:

Enhancers that are specifically active in Schwann cells and not in oligodendrocytes (Lopez-Anido et al., 2015) were identified, including a regulatory element from the Mag intron (LeBlanc et al., 2007; Lopez-Anido et al., 2015). Other studies have identified Schwann cell-specific regulatory elements in the Mbp gene (Denarier et al., 2005) and the Mpz gene (Sargiannidou et al., 2015; Scherer et al., 2005).

ChIP-seq data indicates that functional regulatory elements (e.g., Sox10 binding sites) of the full-length Mpz promoter are located within 400 bp upstream of the start codon (Jang and Svaren, 2009). However, previous studies have shown that the Mpz promoter does not fully recapitulate Mpz expression because it lacks the major binding sites for the Egr2 transcription factor, which is required for high expression levels in myelinating Schwann cells. Therefore, to drive higher levels of expression, we have cloned the intronic Mpz enhancer upstream of the Mpz promoter (SEQ ID NO: 1) in our constructs.

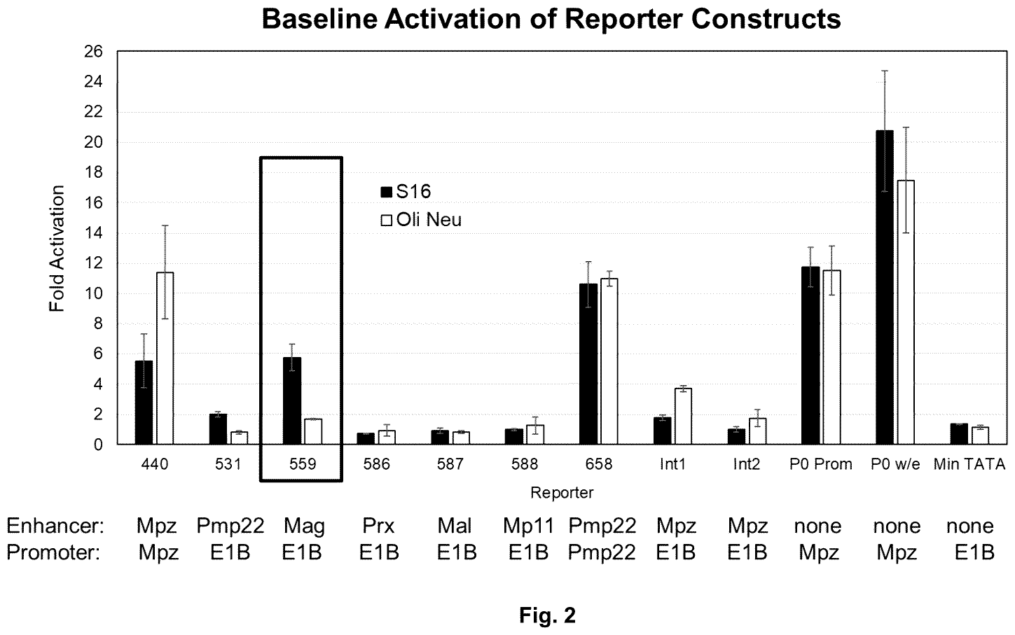

In this Example, we cloned Mag, Mbp, and Mpz regulatory elements upstream of either a minimal promoter (E1B) or a Schwann cell-specific promoter (Mpz) and tested their ability to drive Schwann cell-specific expression using a luciferase assay. While the Mpz/Mpz construct (SEQ ID NO: 1) showed high activity in the transfected Schwann cell line, it was also active in the transfected oligodendrocyte cell line (see construct “440” in FIG. 2). The Mag enhancer (i.e., SEQ ID NO: 3 cloned upstream of the EIB promoter) showed preferential activation in the S16 Schwann cell line (FIG. 2).

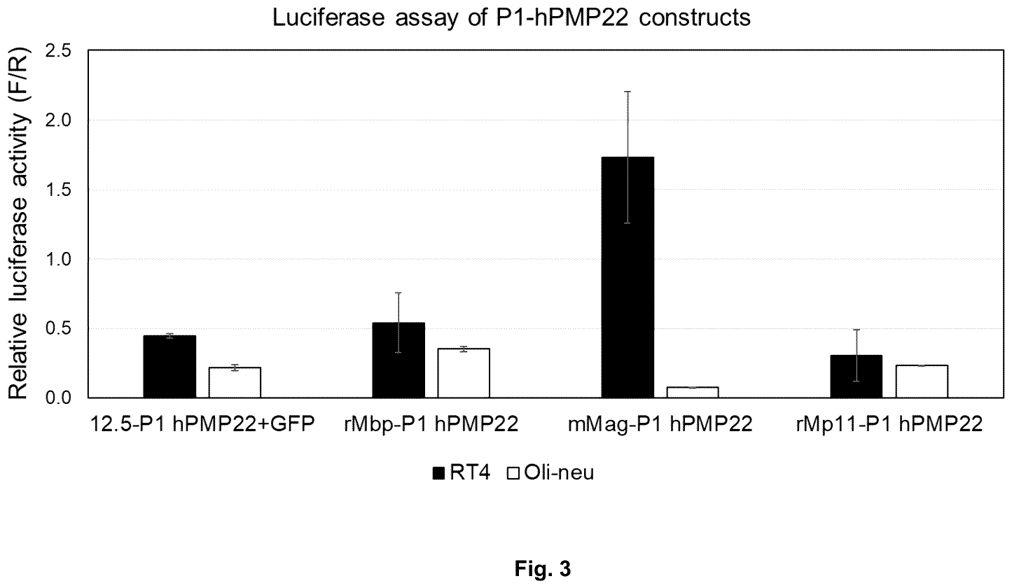

Each of the putative Schwann cell-specific regulatory elements (i.e., Mag, Mp11, Mbp, Pmp22) were also cloned upstream of the Schwann cell-specific, full-length Pmp22 P1 promoter. While several of these constructs showed some degree of preferential activation in the RT4 Schwann cell line, the Mag enhancer (i.e., SEQ ID NO: 3) showed superior preferential activation (FIG. 3).

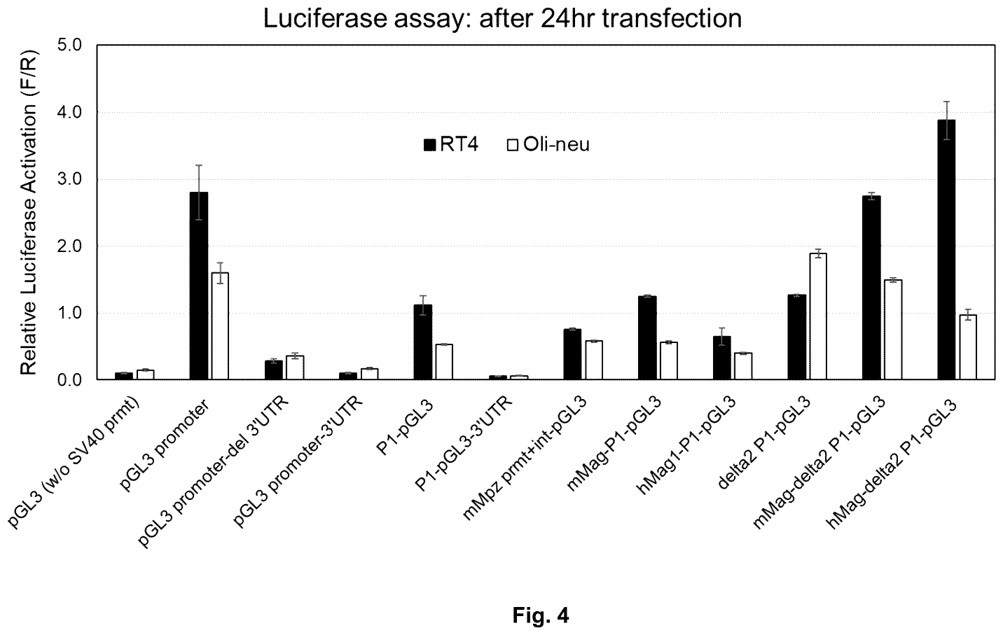

Given the limited insert size that can be used with adeno-associated virus (AAV) vectors, we next tested whether a shorter Pmp22 P1 promoter (referred to as “delta2”) could be used in place of the full-length Pmp22 P1 promoter within our constructs. For this experiment, we cloned the human version of the Mag enhancer (i.e., SEQ ID NO: 2) upstream of both the full-length and short P1 promoters (SEQ ID NO: 7 and SEQ ID NO: 8, respectively). We found that a combination of the human Mag enhancer and the short P1 promoter showed the greatest preferential activation (see hMag-delta2 P1-pGL3 in FIG. 4).

### Aatk Regulatory Elements:

To create a construct that drives Schwann cell-specific expression of PMP22-targeting shRNAs, we tested elements that have been identified as regulators of Schwann cell-specific microRNA expression. Profiling of Sox10-dependent microRNAs that are specifically active in Schwann cells identified two candidates: miR-338 and miR-138 (Gokey et al., 2012). Recently, a small, highly conserved region of the Aatk gene comprising an intronic Sox10 binding sequence was found to drive expression of both the Aatk gene and miR-338. This demonstrates that this Aatk regulatory element can drive Schwann cell-specific expression of microRNAs, making it a good candidate regulator for the expression of shRNAs.

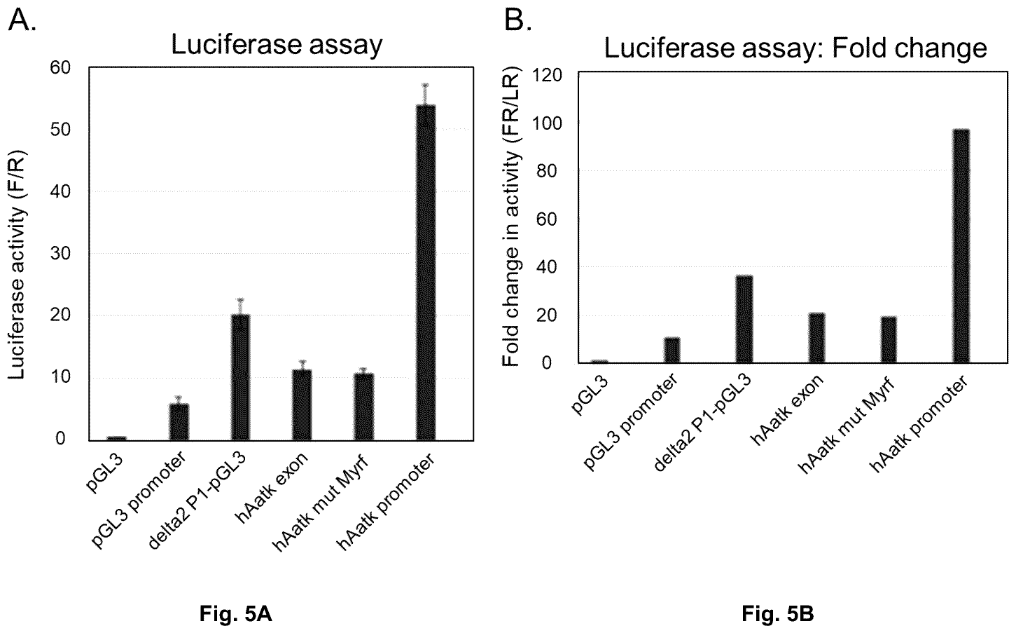

We generated constructs comprising several different versions of the Aatk promoter and tested them in a luciferase assay after transfection into the RT4 cell line. The tested constructs included the pGL3 luciferase reporter vector, the promoter and first Schwann cell-specific exon of Aatk (SEQ ID NO: 6), and the shorterAatk promoter (SEQ ID NO: 7). For comparison, the delta2 PMP22 P1 promoter was also included. Surprisingly, the shorter Aatk promoter construct showed the highest activity in this assay (FIG. 5).

Notably, constructs in which binding sites for the oligodendrocyte-specific transcription factor Myrf were also included in the assay. These mutations did not appear to affect Aatk-driven reporter activity in the RT4 Schwann cell line (FIG. 5). Further mutations of such sites may reduce the activation of the disclosed constructs in oligodendrocytes, thereby improving their Schwann cell-specificity.

### Isoform-Specific Targeting of PMP22:

In addition to designing constructs comprising a shRNA that would target all splice isoforms of PMP22 (i.e., SEQ ID NO: 9-11), we also designed exon-specific shRNAs that specifically target either exon 1a (i.e., SEQ ID NO: 12-14) or exon 1b of this gene. These exon-specific shRNAs target one of two PMP22 splice isoforms that are abundant in the sural nerve (Visigalli et al., 2016), providing targeted, partial silencing of PMP22. The two PMP22 transcript isoforms are expressed in a 1:1 ratio in human nerves. Thus, the maximal reduction of PMP22 expression would be 50% with this approach.

### Further Evaluation:

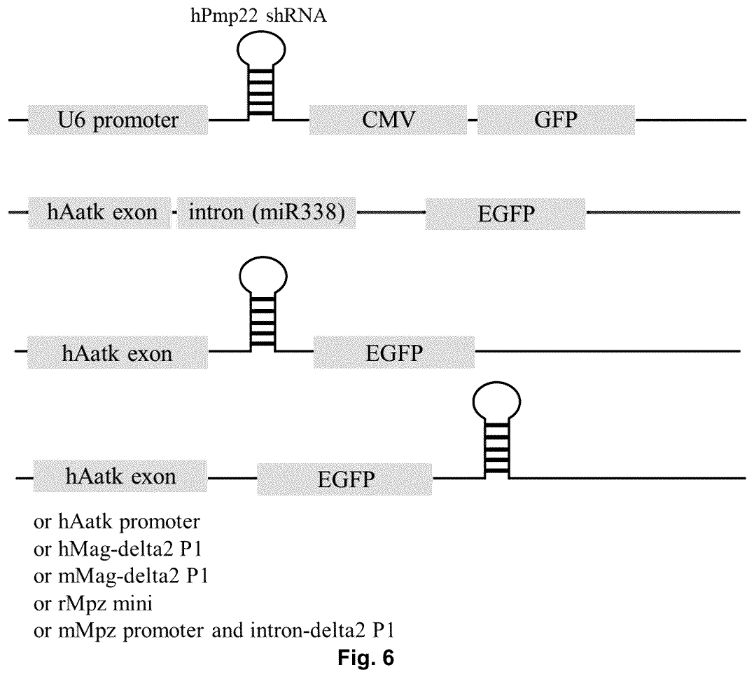

Any minimal promoter elements that generate promising results in the in vitro luciferase assays (e.g., the Mag and Mpz constructs) were next tested in primary human Schwann cells. Successful constructs were then cloned into the adeno-associated virus (AAV) construct pAM/Mbp-EGFP-WPRE-bGH (von Jonquieres et al., 2013). This AAV construct comprises the Egfp reporter gene (FIG. 6), which is used to evaluate the expression driven by the minimal promoter elements in vivo, following injection into mice.

Design of shRNA Expression Cassette Using the miR-3G Format:

A shRNA expression cassette was designed to insert in the 3′ UTR of our constructs based on Watanabe et al. 2016, using the miR-3G format.

The first step is to insert miR flanking sequences using a double strand oligo with the following sequence:

(SEQ. ID. NO: 20)
TCAACGCCCTAGGTTTATGTTTGGATGAACTGACATacgcgttctccaat
tgGCAACTATTTTATCAATTTTTTGCGTCGAC

Appropriate restriction sites are then added to the end(s) to clone into suitable vectors. The shRNA is then cloned into the underlined Mlu1 and Mfe1 sites.

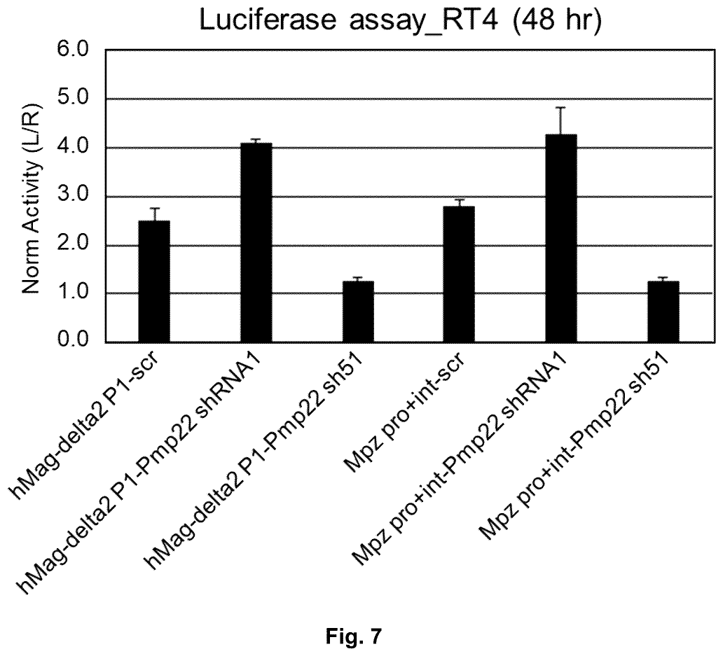

We designed three shRNAs with 5′ UTR for PMP22 (SEQ ID NO: 15-17). PMP22 targeting sites were designed using the SplashRNA algorithm (Pelossof and Fairchild et al., 2017). We then tested the shRNA with 5′ UTR No. 1 (sh51, SEQ ID NO: 15) on a reporter construct containing the 5′ UTR of PMP22 fused to the luciferase genes, and the results showed reduced activity of this construct (FIG. 7). Controls are an shRNA (shRNA1, SEQ ID NO: 18) directed to the coding region of PMP22 (not found in luciferase construct) and also the scrambled shRNA (scr, SEQ ID NO: 19).

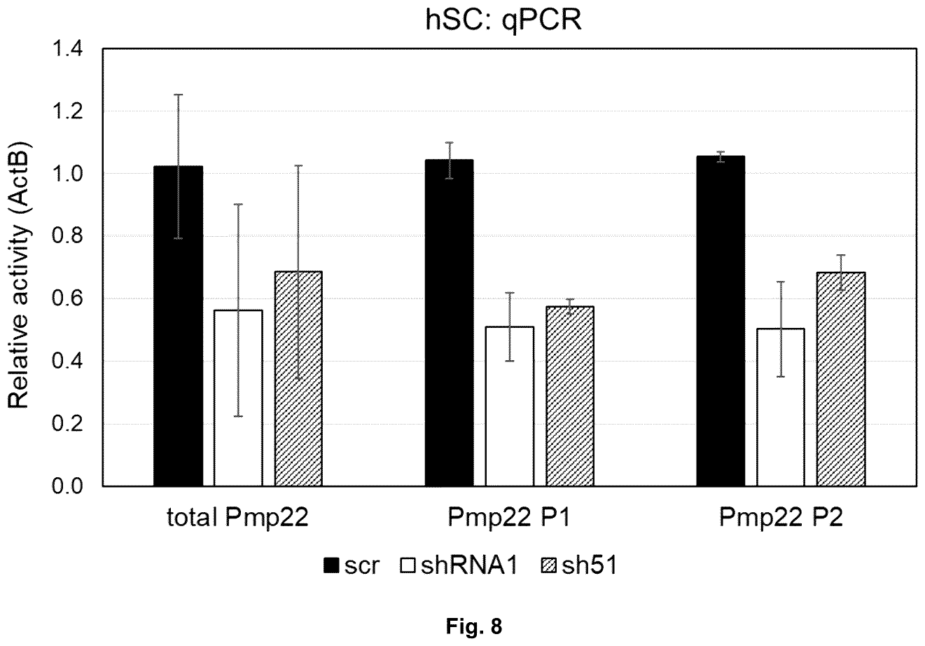

We then transfected human Schwann cell line (hTERT NF1 ipn02.3 2λ, ATCC CRL-3392) with Mpz pro/int vectors expressing sh51, shRNA1 or scr. Transfection efficiency was ˜60%. Results show qPCR analysis of total PMP22 and its two major transcripts (P1 and P2), indicating reduced expression of PMP22 in SC cells expressing sh51 or shRNA1 (FIG. 8).

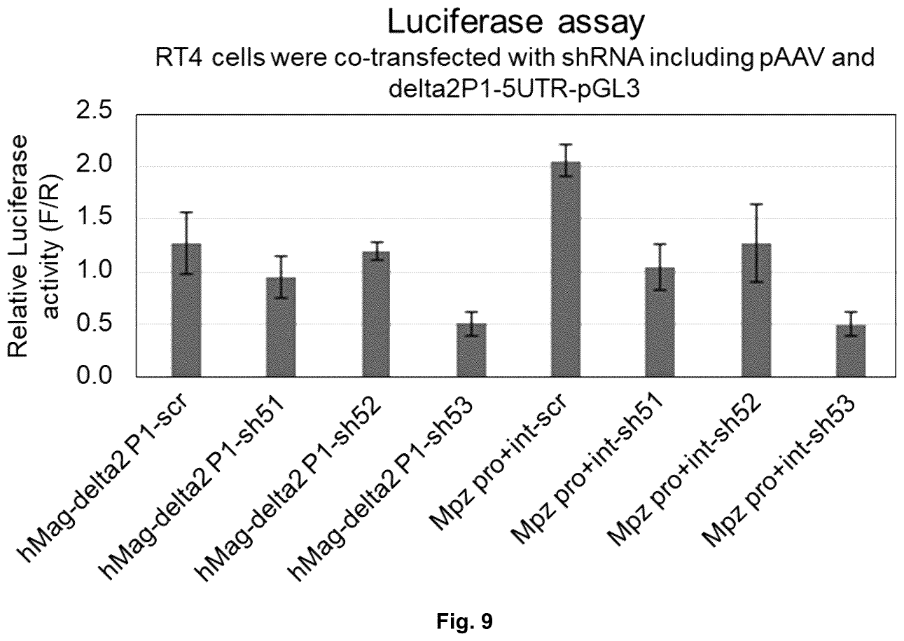

We then cloned the 3 different shRNAs (sh51, sh52, and sh53; SEQ ID NO: 15-17) into the Mag and Mpz vectors and compared their ability to downregulate the PMP22 5′ UTR construct in which luciferase was cloned downstream. These data indicated that sh53 was the best of those cloned into the Mag and Mpz vectors described above (FIG. 9).

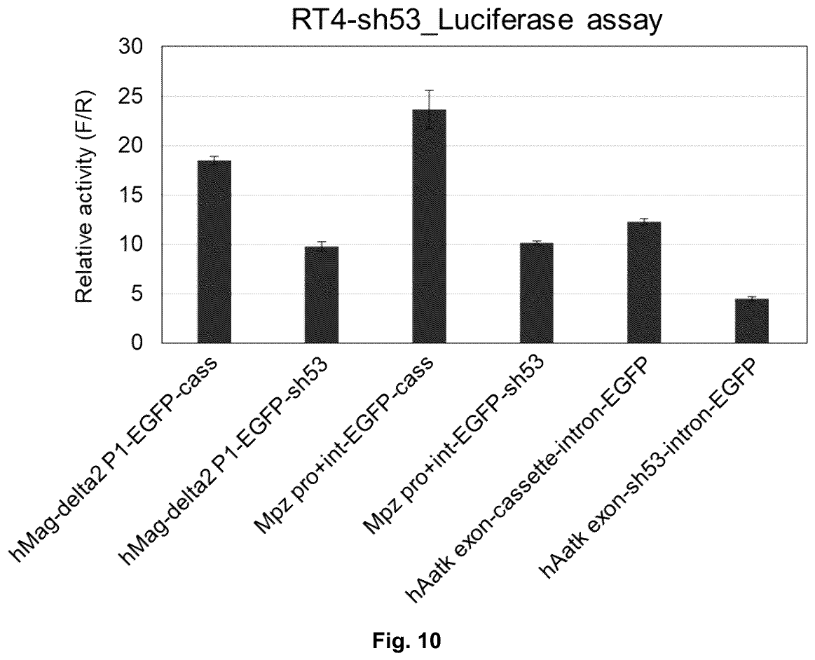

We then cloned sh53 into a variety of different constructs to examine which was the best expression vector for shRNA expression and PMP22 downregulation (FIG. 10).

### REFERENCES CITED

Denarier, E., Forghani, R., Farhadi, H. F., Dib, S., Dionne, N., Friedman, H. C., Lepage, P., Hudson, T. J., Drouin, R., and Peterson, A. (2005). Functional organization of a Schwann cell enhancer. J Neurosci 25, 11210-11217.
Georgiou, E., Sidiropoulou, K., Richter, J., Papaneophytou, C., Sargiannidou, I., Kagiava, A., von Jonquieres, G., Christodoulou, C., Klugmann, M., and Kleopa, K. A. (2017). Gene therapy targeting oligodendrocytes provides therapeutic benefit in a leukodystrophy model. Brain: a journal of neurology 140, 599-616.
Gokey, N. G., Srinivasan, R., Lopez-Anido, C., Krueger, C., and Svaren, J. (2012). Developmental regulation of microRNA expression in Schwann cells. Molecular and cellular biology 32, 558-568.
Jang, S. W., and Svaren, J. (2009). Induction of myelin protein zero by early growth response 2 through upstream and intragenic elements. The Journal of biological chemistry 284, 20111-20120.
Jones, E. A., Brewer, M. H., Srinivasan, R., Krueger, C., Sun, G., Charney, K. N., Keles, S., Antonellis, A., and Svaren, J. (2012). Distal enhancers upstream of the Charcot-Marie-Tooth type 1A disease gene PMP22. Human Molecular Genetics 21, 1581-1591.
Kagiava, A., Sargiannidou, I., Bashiardes, S., Richter, J., Schiza, N., Christodoulou, C., Gritti, A., and Kleopa, K. A. (2014). Gene delivery targeted to oligodendrocytes using a lentiviral vector. The journal of gene medicine 16, 364-373.
Kagiava, A., Sargiannidou, I., Theophilidis, G., Karaiskos, C., Richter, J., Bashiardes, S., Schiza, N., Nearchou, M., Christodoulou, C., Scherer, S. S., et al. (2016). Intrathecal gene therapy rescues a model of demyelinating peripheral neuropathy. Proc Natl Acad Sci USA 113, E2421-2429.
LeBlanc, S. E., Ward, R. M., Svaren, J. (2007). Neuropathy-Associated Egr2 Mutants Disrupt Cooperative Activation of Myelin Protein Zero by Egr2 and Sox10. Mol Cell Biol 27, 3521-3529.
Lopez-Anido, C., Sun, G., Koenning, M., Srinivasan, R., Hung, H. A., Emery, B., Keles, S., and Svaren, J. (2015). Differential Sox10 genomic occupancy in myelinating glia. Glia 63, 1897-1914.
Pelossof, R., Fairchild, L. et al. (2017). Prediction of potent shRNAs with a sequential classification algorithm. Nat. Biotechnology 35, 350-353.
Sargiannidou, I., Kagiava, A., Bashiardes, S., Richter, J., Christodoulou, C., Scherer, S. S., and Kleopa, K. A. (2015). Intraneural GJB1 gene delivery improves nerve pathology in a model of X-linked Charcot-Marie-Tooth disease. Annals of neurology 78, 303-316.
Scherer, S. S., Xu, Y. T., Messing, A., Willecke, K., Fischbeck, K. H., and Jeng, L. J. (2005). Transgenic expression of human connexin32 in myelinating Schwann cells prevents demyelination in connexin32-null mice. The Journal of neuroscience: the official journal of the Society for Neuroscience 25, 1550-1559.
Srinivasan, R., Sun, G., Keles, S., Jones, E. A., Jang, S. W., Krueger, C., Moran, J. J., and Svaren, J. (2012). Genome-wide analysis of EGR2/SOX10 binding in myelinating peripheral nerve. Nucleic Acids Res 40, 6449-6460.
Visigalli, D., Castagnola, P., Capodivento, G., Geroldi, A., Bellone, E., Mancardi, G., Pareyson, D., Schenone, A., and Nobbio, L. (2016). Alternative Splicing in the Human PMP22 Gene: Implications in CMT1A Neuropathy. Hum Mutat 37, 98-109.
von Jonquieres, G., Frohlich, D., Klugmann, C. B., Wen, X., Harasta, A. E., Ramkumar, R., Spencer, Z. H., Housley, G. D., and Klugmann, M. (2016). Recombinant Human Myelin-Associated Glycoprotein Promoter Drives Selective AAV-Mediated Transgene Expression in Oligodendrocytes. Frontiers in molecular neuroscience 9, 13.
von Jonquieres, G., Mersmann, N., Klugmann, C. B., Harasta, A. E., Lutz, B., Teahan, O., Housley, G. D., Frohlich, D., Kramer-Albers, E. M., and Klugmann, M. (2013). Glial promoter selectivity following AAV-delivery to the immature brain. PloS one 8, e65646.
Watanabe C., Cuellar, T. L., Haley, B. (2016). Quantitative evaluation of first, second, and third generation hairpin systems reveals the limit of mammalian vector-based RNAi. RNA Biol 13, 25-33.
Xiang, H., Xu, H., Fan, F., Shin, S. M., Hogan, Q. H., and Yu, H. (2018). Glial fibrillary acidic protein promoter determines transgene expression in satellite glial cells following intraganglionic adeno-associated virus delivery in adult rats. Journal of neuroscience research 96, 436-448.

### SEQUENCE LISTING

Mouse Mpz intron and promoter: mm10
chr1:171,155,287-171,157,034
SEQ ID NO: 1
AGCAAAGATTGACGATATCATAGGAGGAATAGAAATCCAAAGAAATAAGTAACCCTC
TGTGTTACACTGGGGATTCCCAAGAAGGCTCTCAGACAGAATTCCCAGGAGGTAAGT
AAAAGATATGGCATGCTAAGATGGGAGATTCGGGACAATGAGGGGGGAGTGGGTGTG
GTATGAGCCCCCAACATTGGCTAGACAATGGGATTTTGTTTGGTTGCCTCCTCCTAA
AGAACAGAAAAGTCTATAATTGTTCCTACCCAGAGCCACCCCACCCACATAGACTGC
CTGGGATGACTCTCCATGGGTGGGGGGCTTAGAGGGGTGGCACTGGGCCAAGATGAC
CAAGAAGAGGGTGAGGATGGATGGTCGGACCACAGAGGTCCACATGCAGAGGCACCT
GGGTGCTGGACTCGGCATCTTTCCTATATCTAGCTTTCCTTGACAATGAGTCCTTCC
TCCCCTAGATCCCCAGTCATGGTAACCTAGGGTAGGGGGAGGTCAGTATACACAAAG
CCCTCTGTGTAAGGGGTGGTATGTGTCCCCCCACCCCTCCACCCAGCGTATACAATG
CCCCTTCTGCTCCATGCCCCCTGCCACCCTCCCCACCACCTCTCCATTGCACATGCC
AGGCTGCAATTGGTCACTGGCTCAGGACAGCCCCCTCATGCTGGGGATCCAGGGGAT
TTTAAGCAGGTTCCAGAAAACACAGCTCAGTTCCTTGTCCCCCGCTCTCTCCACCCC
ACAGACACTCTGGGCCTTTGCCCTACCC
Human Mag: hg38 chr19:35,293,763-35,293,939
SEQ ID NO: 2
CCCCGAGGGAACAGGACTCTTTTGTACCGGGGACCAGCTTCTGGTGAGGGTGCGGAC
ACCAGGGGCCGTCTGTGGGGTGGGAGAGGGCGGCAGGAATTCACGCGGCATGTCGGG
AATGCTGATACTGTGAAACCCAGTCATTGATGGCTCTGGGTCCCTGCCCGCCACCCC
CAGGCC
Mouse Mag: mm10 chr7:30,912,989-30,913,358
SEQ ID NO: 3
CCTAGGAGGTCTCTAGGATGGGGCACCCACACCCCCAAAGGAACAGGgCTcTTTTGT
ATTGGCTACTGGCCTCTGGTGAGGGTGGGGACACCCTGAGCCTGCTGCGGGTGGGAG
AGGGCGGCAGGAATTCACACGGCATGCTGGGAATGCTGATGTTGTGAAACCCAGTCA
TTGATGGGGCTGGGTCCTTGCCTGCCTGCCACCCCCAGGCCTCATCTGCTCATGGAC
TCACCACTTTCTGTTCCCCTCATCTTAGAGCTACCATTCCCTTCCTTCCCCATGGCA
CTCCCACGCCCCAGCTGGGGCGTGGGGTGTTGTGGTGGTACAGTATCTTTGAAGCTG
TGTGGGAGCATCCTGGGTATCCACGGGTTCTAGCTCG
Rat Mbp: rn5 chr18:78,455,207-78,455,380
SEQ ID NO: 4
CCAGCAGGGGCTAACACTAGCTGACACGGAGCTGACACCATGGGGTGCtCccTTGAT
GGTGGATTGTGGAACTGAAAGACAGCATTGTGTGGGGCCCGATTGCTATTCTCTGCC
AGACATTTGACACCATTGTCCCTGTGGTATCCAGGCACAGTAAGTGCTGCACACAGA
TCT
Human Aatk, exon and promoter: hg38 chr17:81128197-81129281
SEQ ID NO: 5
GTACCTAGCCCCGCCTCTACATTCTCTGGGTCTCCAGGCCCTGGAAGGAGCCCCCTC
CCCCTGGGCTCTCCTGGCAGCCAGGCTCAGACCCAGGCCTGTCAGCCCTCTGGGCAG
TTGACGTGGGAGCAGAATTCGGGGAATCCCTCGGGGCGTGGGCAGGCATGAGTGGGC
TCTGCCAGCGCTGGCCCCTTCGTCCCGGGCCTCACTGACCCTTCCTCCCGCTGACCT
CACCCGGCCTGGCCCTCGGGGAGAATGGGCCAGTGTGCTCCGTGCGGGGCCGTGCCA
AGCAGCCCCCAACAAAGGAGCCTTTGGTGCGCCCGGCGTGGGATGCCAGCCTGCGGC
AGGGTGGGGGTGGGCCGCTCCAGTGCCCTCTCTGTGCCGTGGCCCCAGCCTCTTGCT
CAGCCACACTGCCTGCCTGGTGCAGCCCATGTGACGGGTCGAGCTCCGGGCCCTGCT
GTCCCTGGCCGGGCTATCCCAGTGGCTTCAGGCACCTTCTCCAGACCTACCCAGAAA
GATGCCCGGATGGATCCTGCAGCTCCGTGGCTTTTCTGGGAAGCAGCGGCCCCTGCT
CTCAAGAGACCCTGGCTCCTGATGGTGGCCCCAAGGTTGCCAGCTGGTGCTAGGGAC
TCAGGACAGTTTCCCAGAAAAGGCCAAGCGGGCAGCCCCTCCAGGGGCCGGGTGAGG
AAGCTGGGGGGTGCGGAGGCCACACTGGGTCCCTGAACCCCCTGCTTGGTTACAGTG
CAGCTCCTCAAGTCCACAGACGTGGGCCGGCACAGCCTCCTGTACCTGAAGGAAATC
GGCCGTGGCTGGTTCGGGAAGGTACGTGTCCCGCCTGGGAGGGAGGACTCCCGCCTG
GGAGGCAGAAGGGACACAGGGCTGCAGACCTGCTCCTCCTAGGAGCCACCCCAGCCC
CGACGGTCCCTTCTTCAGTCTCTGGGCCCTGCTGGACCCTTCCCCAGGGAGGTCCTC
AGCATCTGGCTGCATCCCCTTCTCCCTAGGGGGTCCTGGGGTCAGAGGTACCTGGGG
AGGGGCCCTGGGAGGGAGAGGAGCAGCCTCTTGCTCATGGCCTTCCCCTGACCTTGG
CCTAAACGA
Human Aatk, minimized intronic promoter: hg 38
chr17:81128764-81129281
SEQ ID NO: 6
AGCCCCGCCTCTACATTCTCTGGGTCTCCAGGCCCTGGAAGGAGCCCCCTCCCCCTG
GGCTCTCCTGGCAGCCAGGCTCAGACCCAGGCCTGTCAGCCCTCTGGGCAGTTGACG
TGGGAGCAGAATTCGGGGAATCCCTCGGGGCGTGGGCAGGCATGAGTGGGCTCTGCC
AGCGCTGGCCCCTTCGTCCCGGGCCTCACTGACCCTTCCTCCCGCTGACCTCACCCG
GCCTGGCCCTCGGGGAGAATGGGCCAGTGTGCTCCGTGCGGGGCCGTGCCAAGCAGC
CCCCAACAAAGGAGCCTTTGGTGCGCCCGGCGTGGGATGCCAGCCTGCGGCAGGGTG
GGGGTGGGCCGCTCCAGTGCCCTCTCTGTGCCGTGGCCCCAGCCTCTTGCTCAGCCA
CACTGCCTGCCTGGTGCAGCCCATGTGACGGGTCGAGCTCCGGGCCCTGCTGTCCCT
GGCCGGGCTATCCCAGTGGCTTCAGGCACCTTCTCCAGACCTACCCAGAAAGATGCC
CGGATG
Human Pmp22, P1 promoter long: hg38
chr17:15,265,192-15,266,092
SEQ ID NO: 7
ACCTAAATATGCCATAACTTCCTTTACTTCCCTCTCCAAGAATTTCCAATTGCTCTT
CCAGCCTATTCTGCCTGACAGGTTAGAAAGTTTTCAATAGTTTTCAATAGAGGTTCG
TTTACTTGCATTGAATTATCTCATGAAGGTCAGCTTGATTCGGGCACATTAAAGACA
GAGTCGGAAGAACACATTTCCCTAAGTTTTATTCTAATGTACAACACATGTGATCAT
TTGCGATGGGGTCTAGGCAGACAAGGCAGGTAACAGGGAGTCCTTCCAACCAGGGGT
TGGAACAAAGGAGGCTTGATTTGGTGACTCTTGAGACATTTGGCTCACTGCTGTGAT
GCTGTGAGAGATTAGCTGTGCAATGTTTGGGCTCCTTAAAAGGAAGTTTATTTAAAA
TAAAACTTACCTGCACGTATGTAACACTGTAGACACAGATCCTTCCCACAGTATATT
TAATCTCTGCAGAATTCACTGGGAGGGGAGGGGAGCCAGTGGGACCTCTTGGCTATT
ACACAGGTTGGCACTTCCAGAGAGAACAGTCTTGGCATCACAGGCTTCAGGCATACT
CAAAGCTCTTCTCCCTTCTGATTCCAGTTTCTCCATGCCCTGCAGGGCCTCTTGGGA
TTATTGTATTCTGGAAAGCAAACAAAGTTGGACACTGTCTCTTTAAATAATAGAGGC
TGAGAACCTCTCAGGCCACCATGACATATCCCAGCATTGGACCAGCCCCTGAATAAA
CTGGAAAGACGCCTGGTCTGGCTTCAGTTACAGGGAGCACCACCAGGGAACATCTCG
GGGAGCCTGGTTGGAAGCTGCAGGCTTAGTCTGTCGGCTGCGGGTCTCTGACTGCCC
TGTGGGGAGGGTCTTGCCTTAACATCCCTTGCATTTGGCTGCA
Human Pmp22, P1 promoter short: hg38
chr17:15,265,192-15,265,478
SEQ ID NO: 8
GGGCCTCTTGGGATTATTGTATTCTGGAAAGCAAACAAAGTTGGACACTGTCTCTTT
AAATAATAGAGGCTGAGAACCTCTCAGGCCACCATGACATATCCCAGCATTGGACCA
GCCCCTGAATAAACTGGAAAGACGCCTGGTCTGGCTTCAGTTACAGGGAGCACCACC
AGGGAACATCTCGGGGAGCCTGGTTGGAAGCTGCAGGCTTAGTCTGTCGGCTGCGGG
TCTCTGACTGCCCTGGGGGAGGGTCTTGCCTTAaCATCCCTTGCATTTGGCTGCAA
AG
rPmp22 sh1
SEQ ID NO: 9
GATCTCTGTTGACAGTGAGCGACCATTCAACCTCATAGGATAATAGTGAAGCCACAG
ATGTATTATCCTATGAGGTTGAATGGGTGCCTACTGCCTCGGACCGG
rPmp22 sh2
SEQ ID NO: 10
GATCTGCTGTTGACAGTGAGCTGTTATCCTATGAGGTTGAATGGTTTTGGCCACTGA
CTGACTCCATTCCCTCATAGGATAAGAGCCTACTGCCACCGG
mPmp22 sh1
SEQ ID NO: 11
GGAAAGGACGAAACACCGGATTCTTGCTGGTCTGTGCGTGATGAGTGCCTCGAGGCA
CTCATCACGCACAGACCAGCAAGAATTTTTTGGATCCCAAGTTTGTACAAAAAAGCA
GGCTTAA
shRNA targeting exon 1A of hPmp22 (target site 1)
SEQ ID NO: 12
CACCGGGTTACGCTGTTTGGCCGGGCGAACCCGGCCAAACAGCGTAACCC
shRNA targeting exon 1A of hPmp22 (target site 2)
SEQ ID NO: 13
CACCACGCTGTTTGGCCGGGCAGAACGAATTCTGCCCGGCCAAACAGCG
shRNA targeting exon 1A of hPmp22 (target site 3)
SEQ ID NO: 14
CACCGCTGTTTGGCCGGGCAGAAACCGAAGTTTCTGCCCGGCCAAACAGC
shRNA with 5′ UTR no. 1 for PMP22 (sh51)
SEQ ID NO: 15
cgcgtATCCGTCTTTGCAGCCAAATGCAAGGGATgTAGTGAAATATATATTAAACat
cccttgcatttggctgcaaTTACGGTAacgcgc
shRNA with 5′ UTR no. 2 for PMP22 (sh52)
SEQ ID NO: 16
cgcgtATCCGTCTTCCAAGCAGATTTCTTTGCAGGTAGTGAAATATATATTAAACct
gcaaagaaatctgcttggaTTACGGTAacgcgc
shRNA with 5′ UTR no. 3 for PMP22 (sh53)
SEQ ID NO: 17
cgcgtATCCGTCTGTTAAGGCAAGACCCTCCCCAGTAGTGAAATATATATTAAACtg
gggagggtcttgccttaacTTACGGTAacgcgc
shRNA1 for PMP22 coding region
SEQ ID NO: 18
cgcgtATCCGTCTgcactcatcacgcacagacGTAGTGAAATATATATTAAACGTCT
GTGCGTGATGAGTGCTTACGGTAacgcgc
Scrambled shRNA (scr)
SEQ ID NO: 19
cgcgtATCCGTCTCCTAAGGTTAAGTCGCCCTCGTAGTGAAATATATATTAAACgag
ggcgacttaaccttaggTTACGGTAacgcgc
SEQ ID NO: 20
TCAACGCCCTAGGTTTATGTTTGGATGAACTGACATacgcgttctccaattgGCAAC
TATTTTATCAATTTTTTGCGTCGAC

What is claimed:

1. A nucleic acid construct comprising a Schwann cell-specific regulatory element, wherein the regulatory element is operably linked with a gene selected from the group consisting of myelin protein zero (MPZ), myelin-associated glycoprotein (MAG), myelin basic protein (MBP), and apoptosis-associated tyrosine kinase (AATK).

2. The nucleic acid construct of claim 1, wherein the regulatory element comprises at least a portion of a sequence selected from the groups consisting of SEQ ID NO:1-6.

3. The nucleic acid construct of claim 2, wherein the regulatory element is the MAG enhancer of SEQ ID NO:2 or SEQ ID NO:3 or is the minimal AATK promoter of SEQ ID NO:6.

4. The nucleic acid construct of claim 1, further comprising a peripheral myelin protein 22 (PMP22) P1 promoter.

5. The nucleic acid construct of claim 4, wherein the Pmp22 P1 promoter comprises SEQ ID NO:7 or SEQ ID NO:8.

6. The nucleic acid construct of claim 4, wherein the regulatory element comprises at least a portion of a sequence selected from the groups consisting of SEQ ID NO:1-6.

7. The nucleic acid construct of claim 4, wherein the regulatory element is the MAG enhancer of SEQ ID NO:2 or SEQ ID NO:3 or is the minimal AATK promoter of SEQ ID NO:6.

8. The nucleic acid construct of claim 1, wherein the regulatory element is operably linked to a target gene.

9. The nucleic acid construct of claim 8, further comprising a peripheral myelin protein 22 (PMP22) P1 promoter.

10. The nucleic acid construct of claim 9, wherein the Pmp22 P1 promoter comprises SEQ ID NO:7 or SEQ ID NO:8.

11. The nucleic acid construct of claim 8, wherein the regulatory element comprises at least a portion of a sequence selected from the groups consisting of SEQ ID NO:1-6.

12. The nucleic acid construct of claim 8, wherein the regulatory element is the MAG enhancer of SEQ ID NO:2 or SEQ ID NO:3 or is the minimal AATK promoter of SEQ ID NO:6.

13. The nucleic acid construct of claim 8, wherein the target gene is a short hairpin RNA (shRNA) that targets PMP22.

14. The nucleic acid construct of claim 13, wherein the shRNA comprises a sequence selected from SEQ ID NO:9-17.

15. The nucleic acid construct of claim 8, wherein the shRNA is dimensioned, configured, and positioned within the construct to target selectively a single transcript isoform of PMP22.

16. The nucleic acid construct of claim 8, wherein the construct drives expression of the target gene at an attenuated level in oligodendrocytes as compared to expression of the target gene in Schwann cells.

17. A nucleic acid vector comprising the nucleic acid construct of claim 1.

18. A nucleic acid vector comprising the nucleic acid construct of claim 8.

19. A virus particle comprising the nucleic acid construct of claim 1.

20. A virus particle comprising the nucleic acid construct of claim 8.

21. A method of altering gene expression in a Schwann cell, the method comprising delivering the nucleic acid construct as recited in claim 1 to the Schwann cell.

22. The method of claim 21, wherein the nucleic acid construct is delivered by a virus particle.

23. A method of altering gene expression in a Schwann cell, the method comprising delivering the nucleic acid construct as recited in claim 8 to the Schwann cell.

24. The method of claim 23, wherein the nucleic acid construct is delivered by a virus particle.

25. A method of treating a subject having a condition associated with misexpression or attenuated function of a target gene in Schwann cells, the method comprising administering a therapeutically effective amount of a nucleic acid construct as recited in claim 1 to the subject.

26. The method of claim 25, wherein the condition is a peripheral neuropathy.

27. The method of claim 26, wherein the condition is selected from the group consisting of Charcot-Marie-Tooth disease, Guillain-Barré syndrome, schwannomatosis, chronic inflammatory demyelinating polyneuropathy, and leprosy.

28. The method of claim 26, comprising administering a therapeutically effective amount of a nucleic acid construct as recited in claim 1 to the subject, wherein the condition is Charcot-Marie-Tooth disease type 1A (CMT1A), and wherein the target gene is a shRNA that targets PMP22.

29. The method of claim 26, comprising administering a therapeutically effective amount of a nucleic acid construct as recited in claim 1 to the subject, wherein the condition is the X-linked form of Charcot-Marie-Tooth disease (CMT1X), and wherein the target gene is gap junction beta 1 (GJB1).

30. The method of claim 26, comprising administering a therapeutically effective amount of a nucleic acid construct as recited in claim 1 to the subject, wherein the condition is Charcot-Marie-Tooth neuropathy type 4C (CMT4C), and wherein the target gene is SH3 domain and tetratricopeptide repeats 2 (SH3TC2).

31. A method of treating a subject having a condition associated with misexpression or attenuated function of a target gene in Schwann cells, the method comprising administering a therapeutically effective amount of a nucleic acid construct as recited in claim 8 to the subject.

32. The method of claim 31, wherein the condition is a peripheral neuropathy.

33. The method of claim 32, wherein the condition is selected from the group consisting of Charcot-Marie-Tooth disease, Guillain-Barré syndrome, schwannomatosis, chronic inflammatory demyelinating polyneuropathy, and leprosy.

34. The method of claim 32, comprising administering a therapeutically effective amount of a nucleic acid construct as recited in claim 8 to the subject, wherein the condition is Charcot-Marie-Tooth disease type 1A (CMT1A), and wherein the target gene is a shRNA that targets PMP22.

35. The method of claim 32, comprising administering a therapeutically effective amount of a nucleic acid construct as recited in claim 8 to the subject, wherein the condition is the X-linked form of Charcot-Marie-Tooth disease (CMT1X), and wherein the target gene is gap junction beta 1 (GJB1).

36. The method of claim 32, comprising administering a therapeutically effective amount of a nucleic acid construct as recited in claim 8 to the subject, wherein the condition is Charcot-Marie-Tooth neuropathy type 4C (CMT4C), and wherein the target gene is SH3 domain and tetratricopeptide repeats 2 (SH3TC2).


SEQUENCE LISTING (20 sequences)

SEQ ID NO: 1 - DNA, 769 bp, mouse
agcaaagattgacgatatcataggaggaatagaaatccaaagaaataagtaaccctctgtgttacactggggattcccaagaaggctctcagacagaattcccaggaggtaagtaaaagatatggcatgctaagatgggagattcgggacaatgaggggggagtgggtgtggtatgagcccccaacattggctagacaatgggattttgtttggttgcctcctcctaaagaacagaaaagtctataattgttcctacccagagccaccccacccacatagactgcctgggatgactctccatgggtggggggcttagaggggtggcactgggccaagatgaccaagaagagggtgaggatggatggtcggaccacagaggtccacatgcagaggcacctgggtgctggactcggcatctttcctatatctagctttccttgacaatgagtccttcctcccctagatccccagtcatggtaacctagggtagggggaggtcagtatacacaaagccctctgtgtaaggggtggtatgtgtccccccacccctccacccagcgtatacaatgccccttctgctccatgccccctgccaccctccccaccacctctccattgcacatgccaggctgcaattggtcactggctcaggacagccccctcatgctggggatccaggggattttaagcaggttccagaaaacacagctcagttccttgtcccccgctctctccaccccacagacactctgggcctttgccctaccc

SEQ ID NO: 2 - DNA, 177 bp, Homo sapiens
ccccgagggaacaggactcttttgtaccggggaccagcttctggtgagggtgcggacaccaggggccgtctgtggggtgggagagggcggcaggaattcacgcggcatgtcgggaatgctgatactgtgaaacccagtcattgatggctctgggtccctgcccgccacccccaggcc

SEQ ID NO: 3 - DNA, 379 bp, mouse
cctaggaggtctctaggatggggcacccacacccccaaaggaacagggctcttttgtattggctactggcctctggtgagggtggggacaccctgagcctgctgcgggtgggagagggcggcaggaattcacacggcatgctgggaatgctgatgttgtgaaacccagtcattgatggggctgggtccttgcctgcctgccacccccaggcctcatctgctcatggactcaccactttctgttcccctcatcttagagctaccattcccttccttccccatggcactcccacgccccagctggggcgtggggtgttgtggtggtacagtatctttgaagctgtgtgggagcatcctgggtatccacgggttctagctcg

SEQ ID NO: 4 - DNA, 174 bp, rat
ccagcaggggctaacactagctgacacggagctgacaccatggggtgctcccttgatggtggattgtggaactgaaagacagcattgtgtggggcccgattgctattctctgccagacatttgacaccattgtccctgtggtatccaggcacagtaagtgctgcacacagatct

SEQ ID NO: 5 - DNA, 1092 bp, Homo sapiens
gtacctagccccgcctctacattctctgggtctccaggccctggaaggagccccctccccctgggctctcctggcagccaggctcagacccaggcctgtcagccctctgggcagttgacgtgggagcagaattcggggaatccctcggggcgtgggcaggcatgagtgggctctgccagcgctggccccttcgtcccgggcctcactgacccttcctcccgctgacctcacccggcctggccctcggggagaatgggccagtgtgctccgtgcggggccgtgccaagcagcccccaacaaaggagcctttggtgcgcccggcgtgggatgccagcctgcggcagggtgggggtgggccgctccagtgccctctctgtgccgtggccccagcctcttgctcagccacactgcctgcctggtgcagcccatgtgacgggtcgagctccgggccctgctgtccctggccgggctatcccagtggcttcaggcaccttctccagacctacccagaaagatgcccggatggatcctgcagctccgtggcttttctgggaagcagcggcccctgctctcaagagaccctggctcctgatggtggccccaaggttgccagctggtgctagggactcaggacagtttcccagaaaaggccaagcgggcagcccctccaggggccgggtgaggaagctggggggtgcggaggccacactgggtccctgaaccccctgcttggttacagtgcagctcctcaagtccacagacgtgggccggcacagcctcctgtacctgaaggaaatcggccgtggctggttcgggaaggtacgtgtcccgcctgggagggaggactcccgcctgggaggcagaagggacacagggctgcagacctgctcctcctaggagccaccccagccccgacggtcccttcttcagtctctgggccctgctggacccttccccagggaggtcctcagcatctggctgcatccccttctccctagggggtcctggggtcagaggtacctggggaggggccctgggagggagaggagcagcctcttgctcatggccttcccctgaccttggcctaaacga

SEQ ID NO: 6 - DNA, 519 bp, Homo sapiens
agccccgcctctacattctctgggtctccaggccctggaaggagccccctccccctgggctctcctggcagccaggctcagacccaggcctgtcagccctctgggcagttgacgtgggagcagaattcggggaatccctcggggcgtgggcaggcatgagtgggctctgccagcgctggccccttcgtcccgggcctcactgacccttcctcccgctgacctcacccggcctggccctcggggagaatgggccagtgtgctccgtgcggggccgtgccaagcagcccccaacaaaggagcctttggtgcgcccggcgtgggatgccagcctgcggcagggtgggggtgggccgctccagtgccctctctgtgccgtggccccagcctcttgctcagccacactgcctgcctggtgcagcccatgtgacgggtcgagctccgggccctgctgtccctggccgggctatcccagtggcttcaggcaccttctccagacctacccagaaagatgcccggatg

SEQ ID NO: 7 - DNA, 898 bp, Homo sapiens
acctaaatatgccataacttcctttacttccctctccaagaatttccaattgctcttccagcctattctgcctgacaggttagaaagttttcaatagttttcaatagaggttcgtttacttgcattgaattatctcatgaaggtcagcttgattcgggcacattaaagacagagtcggaagaacacatttccctaagttttattctaatgtacaacacatgtgatcatttgcgatggggtctaggcagacaaggcaggtaacagggagtccttccaaccaggggttggaacaaaggaggcttgatttggtgactcttgagacatttggctcactgctgtgatgctgtgagagattagctgtgcaatgtttgggctccttaaaaggaagtttatttaaaataaaacttacctgcacgtatgtaacactgtagacacagatccttcccacagtatatttaatctctgcagaattcactgggaggggaggggagccagtgggacctcttggctattacacaggttggcacttccagagagaacagtcttggcatcacaggcttcaggcatactcaaagctcttctcccttctgattccagtttctccatgccctgcagggcctcttgggattattgtattctggaaagcaaacaaagttggacactgtctctttaaataatagaggctgagaacctctcaggccaccatgacatatcccagcattggaccagcccctgaataaactggaaagacgcctggtctggcttcagttacagggagcaccaccagggaacatctcggggagcctggttggaagctgcaggcttagtctgtcggctgcgggtctctgactgccctgtggggagggtcttgccttaacatcccttgcatttggctgca

SEQ ID NO: 8 - DNA, 287 bp, Homo sapiens
gggcctcttgggattattgtattctggaaagcaaacaaagttggacactgtctctttaaataatagaggctgagaacctctcaggccaccatgacatatcccagcattggaccagcccctgaataaactggaaagacgcctggtctggcttcagttacagggagcaccaccagggaacatctcggggagcctggttggaagctgcaggcttagtctgtcggctgcgggtctctgactgccctgtggggagggtcttgccttaacatcccttgcatttggctgcaaag

SEQ ID NO: 9 - DNA, 104 bp, Artificial Sequence
  rPmp22 sh1
gatctctgttgacagtgagcgaccattcaacctcataggataatagtgaagccacagatgtattatcctatgaggttgaatgggtgcctactgcctcggaccgg

SEQ ID NO: 10 - DNA, 99 bp, Artificial Sequence
  rPmp22 sh2
gatctgctgttgacagtgagctgttatcctatgaggttgaatggttttggccactgactgactccattccctcataggataagagcctactgccaccgg

SEQ ID NO: 11 - DNA, 121 bp, Artificial Sequence
  mPmp22 sh1
ggaaaggacgaaacaccggattcttgctggtctgtgcgtgatgagtgcctcgaggcactcatcacgcacagaccagcaagaattttttggatcccaagtttgtacaaaaaagcaggcttaa

SEQ ID NO: 12 - DNA, 50 bp, Artificial Sequence
  shRNA targeting exon 1A of hPmp22 (target site 1)
caccgggttacgctgtttggccgggcgaacccggccaaacagcgtaaccc

SEQ ID NO: 13 - DNA, 49 bp, Artificial Sequence
  shRNA targeting exon 1A of hPmp22 (target site 2)
caccacgctgtttggccgggcagaacgaattctgcccggccaaacagcg

SEQ ID NO: 14 - DNA, 50 bp, Artificial Sequence
  shRNA targeting exon 1A of hPmp22 (target site 3)
caccgctgtttggccgggcagaaaccgaagtttctgcccggccaaacagc

SEQ ID NO: 15 - DNA, 90 bp, Artificial Sequence
  shRNA with 5′ UTR no. 1 for PMP22 (sh51)
cgcgtatccgtctttgcagccaaatgcaagggatgtagtgaaatatatattaaacatcccttgcatttggctgcaattacggtaacgcgc

SEQ ID NO: 16 - DNA, 90 bp, Artificial Sequence
  shRNA with 5′ UTR no. 2 for PMP22 (sh52)
cgcgtatccgtcttccaagcagatttctttgcaggtagtgaaatatatattaaacctgcaaagaaatctgcttggattacggtaacgcgc

SEQ ID NO: 17 - DNA, 90 bp, Artificial Sequence
  shRNA with 5′ UTR no. 3 for PMP22 (sh53)
cgcgtatccgtctgttaaggcaagaccctccccagtagtgaaatatatattaaactggggagggtcttgccttaacttacggtaacgcgc

SEQ ID NO: 18 - DNA, 86 bp, Artificial Sequence
  shRNA-1 for PMP22 coding region
cgcgtatccgtctgcactcatcacgcacagacgtagtgaaatatatattaaacgtctgtgcgtgatgagtgcttacggtaacgcgc

SEQ ID NO: 19 - DNA, 88 bp, Artificial Sequence
  Scrambled shRNA (scr)
cgcgtatccgtctcctaaggttaagtcgccctcgtagtgaaatatatattaaacgagggcgacttaaccttaggttacggtaacgcgc

SEQ ID NO: 20 - DNA, 82 bp, Artificial Sequence
  Micro RNA (miR) flanking sequence
tcaacgccctaggtttatgtttggatgaactgacatacgcgttctccaattggcaactattttatcaattttttgcgtcgac